(19) United States
(12) Patent Application Publication
(10) Pub. No.: US 2011/0196305 A1
(43) Pub. Date: Aug. 11, 2011

(54) FLOW RATE CONTROL APPARATUS AND PUMP APPARATUS

(76) Inventor: Seiichi Katoh, Miyagi (JP)

(21) Appl. No.: 12/983,680

(22) Filed: Jan. 3, 2011

(30) Foreign Application Priority Data
Feb. 5, 2010 (JP) 2010-024256

(51) Int. Cl. A61M 5/168

(52) U.S. Cl. 604/151; 604/246

(57) ABSTRACT

A disclosed flow rate control apparatus includes a first substrate, a second substrate partially bonded to the first substrate, and a piezoelectric material. The first substrate includes a separation section separating first and second flow paths in the first substrate, the piezoelectric material is adhered to an upper surface of the second substrate above the separation section, and the first substrate is not bonded to the second substrate near the separation section.

### CROSS-REFERENCE TO RELATED APPLICATIONS

The present application claims priority under 35 U.S.C §119 based on Japanese Patent Application No. 2010-024256 filed Feb. 5, 2010, the entire contents of which are hereby incorporated herein by reference.

### BACKGROUND OF THE INVENTION

1. Field of the Invention

The present invention generally relates to a flow rate control apparatus and a pump apparatus. More particularly, the present invention relates to a flow rate control apparatus and a pump apparatus preferably adapted to adjust an injection amount (flow rate) of a medical solution to be injected from a container accommodating the medical solution into a living body.

2. Description of the Related Art

Generally, an infusion apparatus is used when it is necessary to inject a medical solution into a living body. In such an injection apparatus, one end of a tube is connected to a container (transfusion container) accommodating a medical solution, and the other end of the tube is connected to an injection needle so that the medical solution can be injected into a living body. Further in the middle of the tube, there is provided a flow rate control apparatus to adjust the injection rate of the medical solution.

In the description, it is assumed that the term “living body” is not limited to human beings (human bodies) but broadly includes bodies of animals; that the term “into a living body” broadly includes into a vein and an organ of the living body; and that the term “medical solution” (which may also be called “transfusion”) broadly includes a liquid medicine, and a liquid to be injected into a living body via a tube and the like.

Conventionally, to control the flow rate of a medical solution to be injected into a living body, a method using a device including an infusion tube and a clamp is widely used. In most cases, a medical staff member such as a nurse may operate the clamp while watching a drip state of the medical solution in the infusion tube. However, in this method, an operator such as the medical staff member may have to adjust the clamp based on his/her experiences and/or intuition.

More specifically, the operator may have to determine the drip state of the medical solution by carefully observing the size of the liquid droplets dropping in the infusion tube and the number of the liquid droplets per unit time period observed. Because of this feature, in this method, it may be difficult especially for a less-experienced operator to determine an appropriate flow rate of a medical solution to be injected into a living body.

Besides the above method, there is a known apparatus which is called an infusion pump. In the infusion pump, an appropriate flow rate (a flow amount per unit time period, i.e., injection amount) is adjusted by using a motor to drive a syringe, the motor having a mechanism in which the number of revolutions can be controlled or by using a peristaltic pump that presses a tube.

For example, Japanese Patent Application Publication No. 2002-126092 (Patent Document 1) discloses a self-contained type transfusion system capable of controlling a dose (injection) amount of a medical solution such as insulin to be injected at lower flow rates. In the transfusion system, to control the flow rate, a piezoelectric valve is operated to be opened and closed periodically and feedback control is performed using a signal from a thermal flow rate sensor.

However, the technique described in Patent Document 1 is adapted to control a smaller amount of medical solution such as a case of dosing insulin. Further, due to the configuration of the transfusion system, the transfusion system is used for normal (small) transfusions. Because of this limitation, the technique described in Patent Document 1 may be difficult to be adapted to control the flow rate ranging, for example, from 100 ml/hr to 300 ml/hr. Further, in the technique described in Patent Document 1, a plastic film is used as the valve and the configuration of the system becomes complicated. Therefore, it may be difficult to assemble the system.

### SUMMARY OF THE INVENTION

The present invention is made in light of the above circumstances, and may provide a flow rate control apparatus and a pump apparatus capable of adjusting a flow rate to up to a desired flow rate usually set in normal (typical) transfusions with a simpler configuration.

According to an aspect of the present invention, there is provided a flow rate control apparatus to be provided in a middle of a medical solution injection path from a container accommodating medical solution into a living body and capable of adjusting a flow rate of the medical solution. The flow rate control apparatus includes a first substrate; a second substrate, at least a part of the second substrate being bonded to the first substrate; and a piezoelectric material. In the flow rate control apparatus, the first substrate includes a separation section having a thickness equivalent to a thickness of the first substrate where the first substrate is bonded to the second substrate; first and second flow paths are formed in the first substrate, the first flow path and the second flow path being separated from each other by the separation section; the piezoelectric material is adhered to a position on a surface of the second substrate, the position corresponding to a position of the separation section, the surface being opposite to a surface facing the first substrate; and the first substrate is not bonded to the second substrate near the separation section.

### BRIEF DESCRIPTION OF THE DRAWINGS

Other objects, features, and advantages of the present invention will become more apparent from the following description when read in conjunction with the accompanying drawings, in which:

FIG. 1 is a schematic drawing illustrating a configuration of a flow rate control apparatus according to an embodiment of the present invention;

FIG. 2 is a schematic drawing illustrating the configuration of the flow rate control apparatus when a control flow path is opened;

FIG. 3A is a top view of a first substrate in an area defined by a dotted square of FIG. 1;

FIG. 3B is a top view of the flow rate control apparatus in the area defined by the dotted square of FIG. 1;

FIG. 4A is a cross-sectional view cut long line A-B of FIG. 3B;

FIG. 4B is a cross-sectional view cut long line A-B of FIG. 3B when the control flow path is opened;

FIG. 4C is a cross-sectional view cut long line C-D of FIG. 3B;

FIG. 4D is a cross-sectional view cut long line E-F of FIG. 3B;

FIG. 5 is a drawing illustrating drive voltage waveforms applied by voltage application means;

FIG. 6 is a drawing schematically illustrating a configuration of another flow rate control apparatus according an embodiment of the present invention;

FIG. 7A is a cross-sectional view cut long line A-B of FIG. 6;

FIG. 7B is a cross-sectional view cut long line A-B of FIG. 6 when the control flow path is opened;

FIG. 8A is a schematic cross-sectional view of a pump apparatus in a non-driven state according to an embodiment of the present invention;

FIG. 8B is a schematic cross-sectional view of the pump apparatus when liquid is being supplied to a liquid chamber;

FIG. 8C is a schematic cross-sectional view of the pump apparatus when liquid is being discharged from the liquid chamber;

FIG. 9 is an schematic drawing of drive voltage waveforms applied by the voltage application means of the pump apparatus; and

FIG. 10 is a schematic drawing illustrating an exemplary configuration of a medical solution injection system according to an embodiment of the present invention.

### DETAILED DESCRIPTION OF THE PREFERRED EMBODIMENTS

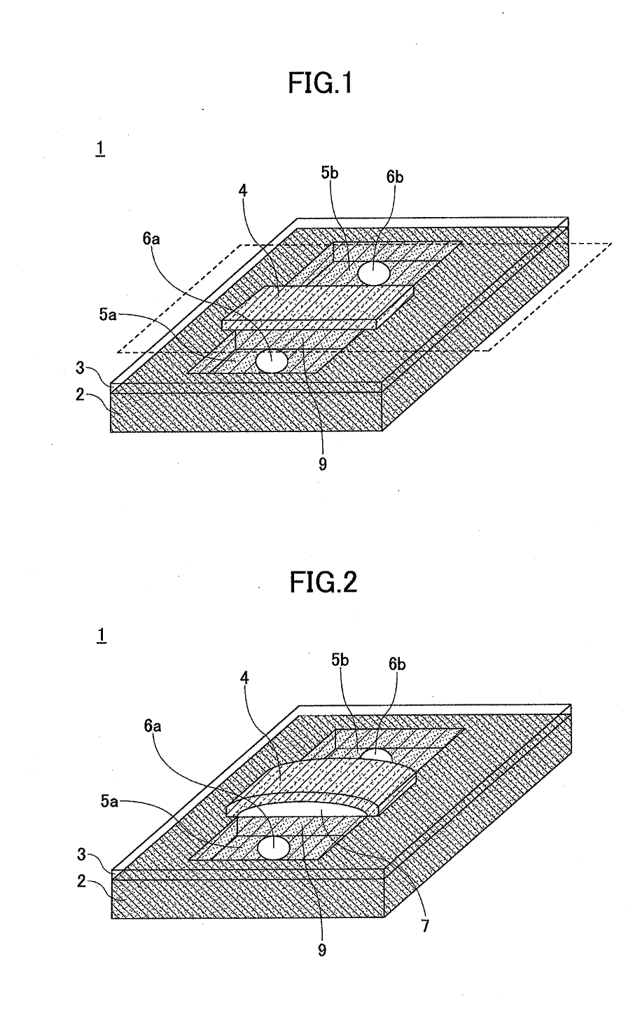

In the following, exemplary configurations according to an embodiment of the present invention are described based on embodiments of the present invention with reference to FIGS. 1 through 10.

### Flow Rate Control Apparatus

A flow rate control apparatus according to an embodiment of the present invention is connected (disposed) at the middle of a medical solution injection path through which a medical solution flows from a container accommodating the medical solution and is injected into a living body.

Further, as schematically illustrated in FIG. 1, a flow rate control apparatus 1 according to an embodiment of the present invention includes a first substrate 2, a second substrate 3, and a piezoelectric material 4. At least a part of the second substrate 3 is bonded to the first substrate 2. The first substrate 2 includes a separation section formed in a manner such that the separation section 9 has a thickness (height) equivalent to the thickness of the first substrate 2 where the first substrate 2 is bonded to the second substrate 3. Further, a first flow path 5a and a second flow path 5b are formed (defined) in the first substrate 2 in a manner such that the first flow path 5a and the second flow path 5b are separated from each other by the separation section 9. Further, the piezoelectric material 4 is adhered to (provided on) an area on a surface of the second substrate 3, the surface being opposite to the surface facing the first substrate 2, and the area corresponding to the separation section 9 of the first substrate 2. Further, the first substrate 2 and the second substrate 3 are not bonded to each other in the area near the separation section 9.

More detail of the flow rate control apparatus 1 according to an embodiment of the present invention is described with reference to FIGS. 1 through 4.

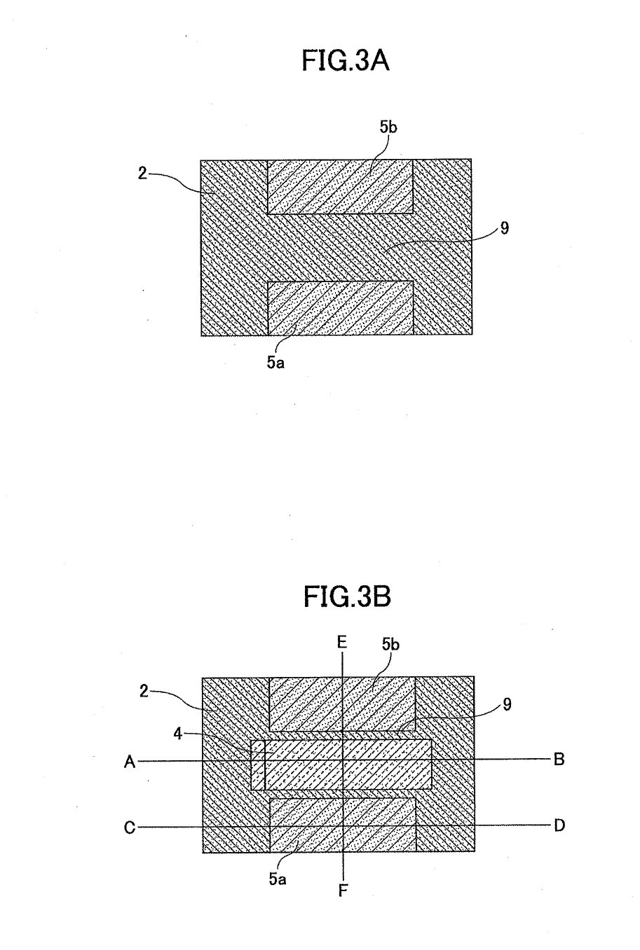

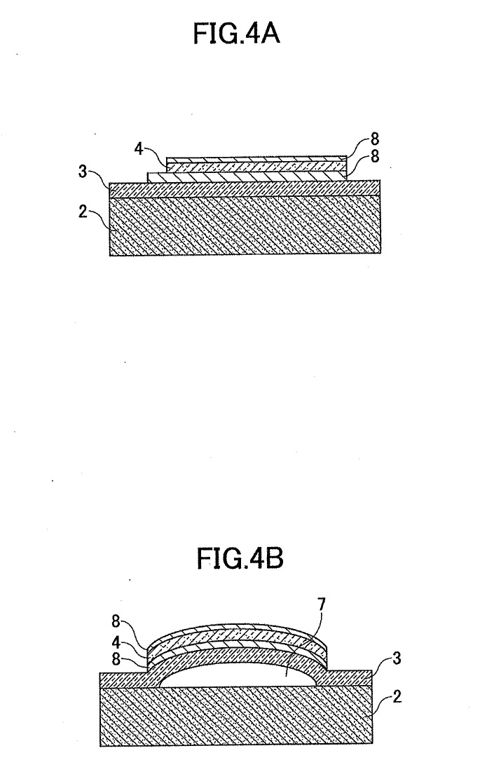

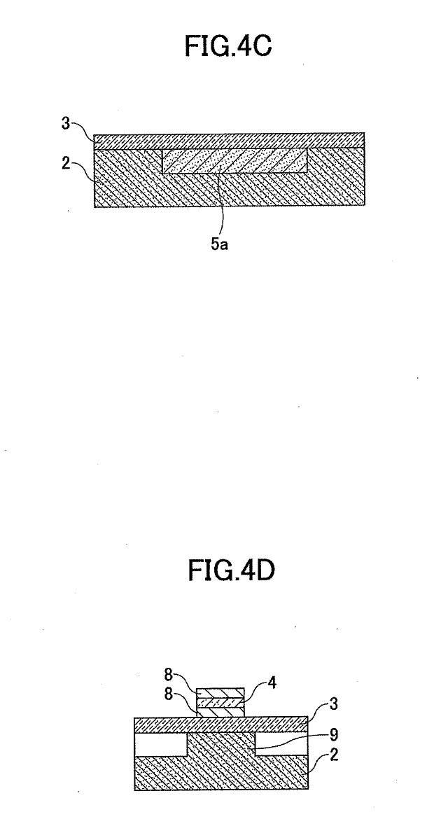

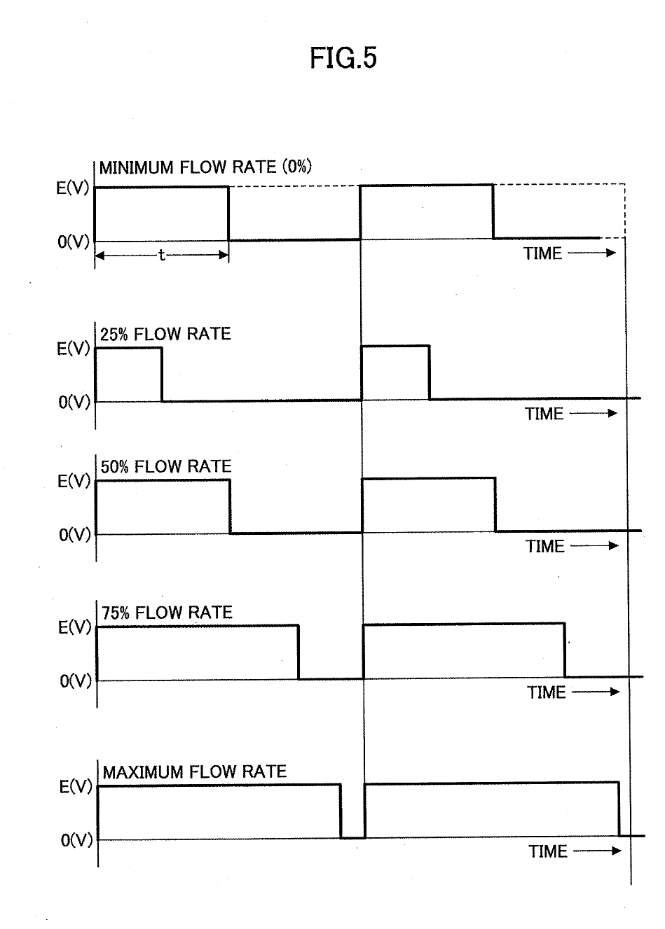

FIG. 1 schematically illustrates an exemplary configuration of the flow rate control apparatus 1. FIG. 2 illustrates a status where a control flow path is opened (described below) of the flow rate control apparatus 1 of FIG. 1. FIGS. 3A and 3B are top views of the area indicated by the dotted lines (square) of FIG. 1. FIGS. 4A through 4D are cross-sectional views cut along the corresponding lines illustrated in FIG. 3B.

As illustrated in FIG. 1 and as described above, groove-shaped flow paths 5a and 5b serving as flow paths to perform transfusions are formed in the first substrate 2. Further, through holes 6a and 6b are formed through the first substrate 2 so that the flow paths 5a and 5b are in communication with the outside of the first substrate 2 through the through holes 6a and 6b, respectively.

Further, as described above, a middle part (i.e., separation section 9, detail is described below) formed between the flow path 5a and the flow path 5b along the longitudinal direction of the flow paths 5a and 5b is formed so as to have a thickness equivalent to the thickness of the first substrate 2 surrounding the middle part.

Preferably, the first substrate 2 may be, for example, a single-crystal silicon substrate. Further, the flow paths 5a and 5b and the through holes 6a and 6b may be formed by performing, for example, photolithography and etching on the silicon substrate.

As described above, the second substrate 3 is partially bonded to the first substrate 2. Preferably, the second substrate 3 may be, for example, made of borosilicate glass having a coefficient of thermal expansion equivalent to that of silicon. Further, preferably, the first substrate 2 and the second substrate 3 may be bonded to each other based on, for example, anodic bonding.

In this case, specifically, a polished surface of the borosilicate glass substrate to be bonded and a polished surface of the silicon substrate to be bonded are in contact with each other, and heat and voltage are applied to the contacting surfaces so as to generate covalent bonding. As a result, those surfaces of first and second substrates are strongly bonded to each other.

Further, as described above, the piezoelectric material 4 is adhered to the area on a surface of the second substrate 3, the surface being opposite to a surface facing (being contact with) the first substrate 2, the area corresponding to the separation section 9 (i.e., above the separation section 9 in FIG. 1). As the piezoelectric material 4, it may be preferable to use PZT (Lead Zirconate Titanate).

Though it is not shown in FIG. 1, an electrode 8 is formed on the second substrate 3 (between the second substrate 3 and the piezoelectric material 4), and another electrode 8 is also formed on a surface of the piezoelectric material 4, the surface being opposite to the surface on the second substrate 3 side (see FIG. 4). The electrodes 8 may be formed based on the sputtering method, the evaporation method or the like. Further, the electrodes 8 are connected to external voltage application means (not shown).

Regarding the bonding between the first substrate 2 and the second substrate 3, due to the anodic bonding or the like, the first substrate 2 is strongly bonded to the second substrate 3 in the (contacting) area other than the area which is under the piezoelectric material 4 (i.e., the area other than the area near the separation section 9). In other words, in the area near (above) the separation section 9, there is in a state where the first substrate 2 and the second substrate 3 are not bonded to each other unlike the other area where the first substrate 2 and the second substrate 3 are strongly bonded to each other based on, for example, anodic bonding.

Herein, the term “the state where the first substrate 2 and the second substrate 3 are not bonded to each other” refers to a not only where the first substrate 2 is in contact with the second substrate 3 but also where there is temporary adhesion between the first substrate 2 the second substrate 3, but when the second substrate 3 is bent due to displacement (bent) of the piezoelectric material 4, the adhesion is released. Further, the term an area “near the separation section 9” does not always include the entire separation section 9. Namely, the area “near the separation section 9” refers to an area which is determined so that when a control flow path 7 is formed due to the bending of the second substrate 3 (described below), the control flow path 7 establishes communication between the flow path 5a and the flow path 5b.

The configuration may be achieved by, for example, forming an adhesion area where the first substrate 2 is temporary adhered to the second substrate 3 near the separation section 9. To achieve the temporary adhesion, the surfaces of the adhesion area may be formed as a rough surfaces by etching or the like. Otherwise, for example, the temporary adhesion may be achieved by inserting a different material such as a silicon oxide film, a silicon nitride film or the like between the first substrate 2 and the second substrate 3 in a process of performing the anodic bonding or the like between the second substrate 3 and the first substrate 2.

By having the configuration of the flow rate control apparatus 1, when a voltage is applied by the voltage application means via the electrodes 8 to the piezoelectric material 4 which is adhered to the second substrate 3, it may become possible to displace (bend) the piezoelectric material 4 in the d31 direction as illustrated in FIG. 2. In this case, the piezoelectric material 4 extends its length in its longitudinal direction.

Further, as described above, near the separation section 9, there is an area where the first substrate 2 and the second substrate 3 are not bonded to each other. As a result, due to the displacement (bending) of the piezoelectric material 4, the second substrate 3 which is adhered to the piezoelectric material 4 is accordingly displaced in the longitudinal direction, thereby bending the second substrate 3.

FIG. 2 schematically illustrates the flow rate control apparatus 1 where the second substrate 3 is bent (displaced). Due to the bending of the second substrate 3, the first substrate 2 and the second substrate 3 are separated from each other. As a result of the separation, a gap (space) (i.e., the control flow path 7) is formed between the first substrate 2 and the second substrate 3.

For example, a case is described where the flow rate control apparatus 1 having the configuration as illustrated in FIGS. 1 and 2 is disposed in a manner such that the near side in the figures (where the flow path 5a and the through hole 6a are formed) is disposed at a position higher in the vertical direction (higher pressure) than the position (lower pressure) of the far side in the figures (where the flow path 5b and the through hole 6b are formed).

In this case, by changing the status of the flow rate control apparatus 1 from a state where no voltage is applied as illustrated in FIG. 1 (which may be referred to as “a non-application state”) to a state where a voltage is being applied as illustrated in FIG. 2 (which may be referred to as “a voltage application state”), it may become possible to cause a medical solution or the like to flow from the through hole 6a to the through hole 6b via the flow path 5b, the control flow path 7, and the flow path 5b in this order.

As described above, by having (forming) the control flow path 7, it may become possible to change (vary) the fluid resistance of a medical solution in the flow rate control apparatus 1, thereby enabling controlling the flow rate of the medical solution. In other words, the first substrate 2 (more specifically, the separation section 9) and the part of the second substrate 3 corresponding to (facing) the separation section 9 may serve as a valve.

Specifically, in the non-application state, the first substrate 2 (i.e., the separation section 9) is in contact with the second substrate 3 (i.e., the valve is closed (“closed valve state”)) and the fluid resistance is higher. Next, a voltage is applied to displace (deform) the second substrate 3 to expand the gap formed between the first substrate 2 and the second substrate 3 (i.e., the valve is opened (“opened valve state”)). As a result, the fluid resistance may be reduced.

In this case, needless to say, it is not always necessary that a value of the flow rate be set to “0” in the non-application state. It may be applicable as long as at least a predetermined difference can be generated (obtained) between the non-application state and the voltage application state.

Further, in this embodiment, an etching process has been performed on the area corresponding to the separation section 9 of the first substrate 2, so that a small flow rate may be generated from the side having higher pressure to the side having lower pressure.

Further, in the flow rate control apparatus 1 according to this embodiment of the present invention, the flow path that may be in contact with a medical solution is exclusively made of the single-crystal silicon substrate 2 or the borosilicate glass substrate 3. Because of this feature, no organic matter may be dissolved into the medical solution and high safety may be achieved. Further, in case of an abnormal condition such as electric power failure, a transfusion flow may be better controlled and high safety may also be achieved.

As an example of the flow rate control apparatus 1, the thickness of the borosilicate glass substrate 3, and the thickness of the PZT-5A material as the piezoelectric material 4 are set to 150 μm and 0.2 mm, respectively, and the sizes of the flow rate control apparatus 1 are set to 2 mm (lateral) by 10 mm (vertical).

In this configuration, when a drive voltage 70 V is applied, a displacement of 4 μm is obtained. Further, under the condition that the height difference between the transfusion container and the flow rate control apparatus 1 is 70 cm, the flow rates of 400 ml/hr and 40 ml/hr in the “opened valve state” in FIG. 2 and the “closed valve state” in FIG. 1, respectively, are obtained.

### Duty Control

As described above, in a flow rate control apparatus 1 according to an embodiment of the present invention, by performing control to select one of no voltage being applied and voltage being applied, a state where a valve is closed and a state where the valve is opened, respectively, may be generated. In other words, the fluid resistance may be changed by performing a two-value control to generate the “closed valve state” and the “opened valve state”.

Because of this feature, by performing a further control of adjusting the time periods of the “closed valve state” and the “opened valve state”, it may become possible to acquire a desired flow rate. In this case, the flow rate may be changed depending on the height difference between the transfusion container and the flow rate control apparatus 1. Therefore, it may be necessary to consider the height difference when controlling the time periods to acquire a desired flow rate.

FIG. 5 schematically illustrates drive voltage waveforms (representing respective duty ratios D) to be applied to the electrodes 8 of the flow rate control apparatus 1, the voltage waveforms being generated by the voltage application means. The duty ratio D is a ratio of the pulse width (i.e., ratio of a high level period to a single cycle period). By changing the duty ratio D by applying a drive pulse having a predetermined cycle period, it may become possible to increase (change) a time period “t” of the “opened valve state”, thereby enabling increasing the flow rate.

FIG. 5 illustrates cases of the flow rates of the minimum flow rate (0%), 25%, 50%, 75%, and the maximum flow rate (100%). For example, when assuming that the drive cycle is 2000 Hz, by changing the duty ratio D, it may become possible to control the flow rate in a range from 50 ml/hr (as the minimum flow rate) to 300 ml/hr (as the maximum flow rate).

In the above description, a method is described in which the flow rate is controlled by changing the duty ratio D while the voltage is constant. However, the control method of controlling the flow rate is not limited to the method. For example, a desired flow rate may be obtained by changing (adjusting) the flow rate based on the pulse interval modulation (with a constant voltage) in which the pulse interval between the pulse widths having a constant width is changed. Otherwise, a desired flow rate may be obtained by changing (adjusting) the flow rate by changing the applied voltage (drive voltage) value to change the displacement amount. Further, for example, a desired flow rate may be obtained by any combination of the above-described methods of controlling the flow rate.

Further, for example, to obtain a desired flow rate, plural flow rate control apparatuses 1 may be connected in series or in parallel in the flow path(s) of the transfusion.

In the flow rate control apparatuses 1 according to this embodiment of the present invention described above, the single-crystal silicon substrate 2 and the borosilicate glass substrate 3 may be used as the components of the valve, and an accurate valve function may be obtained with a simple configuration.

Further, by controlling the time period to open the valve per a constant cycle period, it may become possible to accurately control the flow rate. Further, the control may be performed in a cycle period ranging from several kHz to several tens of kHz. By doing this, it may become possible to prevent the occurrence of pulsation phenomena in the controlled flow.

Further, similar to a micro pump, the flow rate control apparatus may be fabricated by using the MEMS (Micro Electro Mechanical Systems) technique. Therefore, it may become possible to fabricate the flow rate control apparatus at a low cost. Further, the piezoelectric material 4 is used to generate the driving force to bend the second substrate 3. Therefore, it may become possible to increase the speed of opening and closing the valve, thereby enabling an accurate flow rate control.

### Second Embodiment

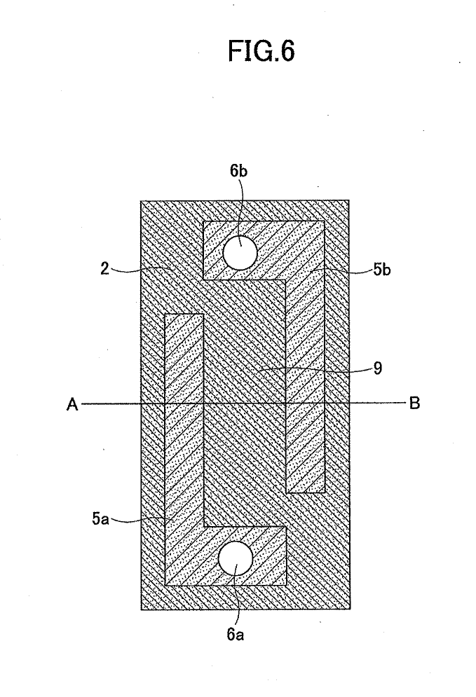

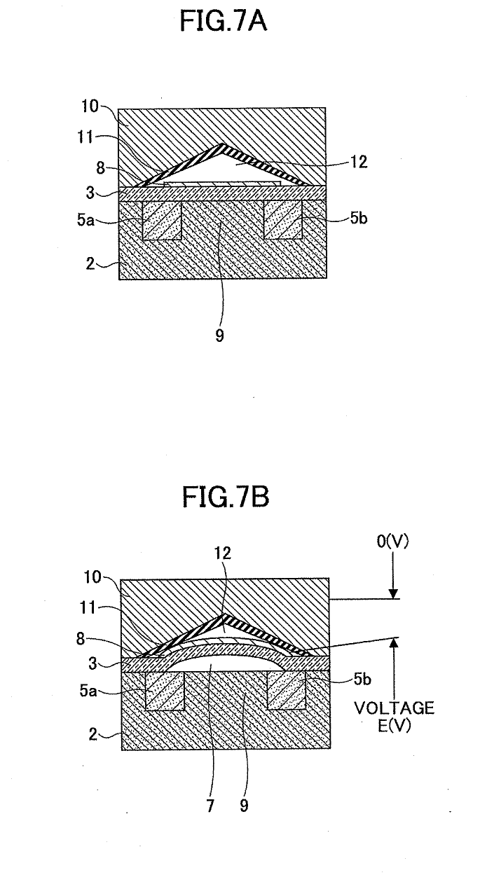

Next, a flow rate control apparatus according to another (second) embodiment of the present invention is described with reference to FIGS. 6 through 7B. The flow rate control apparatus according to this embodiment of the present invention is connected (disposed) at the middle of a medical solution injection path through which a medical solution flows from a container accommodating the medical solution and is injected into a living body.

Further, as schematically illustrated in FIGS. 6 through 7B, a flow rate control apparatus 1 according to this embodiment of the present invention includes a first substrate 2, a second substrate 3, a third substrate 10 and the piezoelectric material 4. At least a part of the second substrate 3 is bonded to the first substrate 2. The first substrate 2 includes a separation section 9 which is formed in a manner such that the separation section 9 has a thickness (height) equivalent to a thickness of the first substrate 2 where the first substrate 2 is bonded to the second substrate 3. Further, a first flow path 5a and a second flow path 5b are formed (defined) in the first substrate 2 in a manner such that the first flow path 5a and the second flow path 5b are separated from each other by the separation section 9.

Further, an electrode 8 is formed at a position on a surface of the second substrate 3, the surface of the second substrate 3 being opposite to the surface facing the first substrate 2, the position corresponding to the position of the separation section 9 of the first substrate 2, and the position being opposite to an electrode of the third substrate 10 via a gap (space) 12. Further, the first substrate 2 and the second substrate 3 are not bonded to each other near the separation section 9.

FIG. 6 is a top view of the first substrate 2. FIGS. 7A and 7B are cross-sectional views cut along the line A-B of FIG. 6. In the description of this second embodiment, the repeated descriptions of the same elements as those described in the first embodiment may be omitted.

As schematically illustrated in FIG. 6, the flow paths 5a and 5b and the through holes 6a and 6b are formed by performing photolithography and etching on a single-crystal silicon substrate as the first substrate 2.

Further, as schematically illustrated in FIGS. 7A and 7B, the first substrate 2 is bonded to the borosilicate glass substrate as the second substrate 3 based on, for example, anodic bonding.

Further, in this embodiment, there is formed an area where the first substrate 2 is not (strongly) bonded to the second substrate 3 so that the control flow path 7 can be generated (formed) to bridge (communicate) between the first flow path 5a and the second flow path 5b when the second substrate 3 is bent (as described below). Further, the electrode 8 made of aluminum or the like is formed on the (upper) surface of the second substrate 3 and is connected to external voltage application means (not shown).

Further, as schematically illustrated in FIGS. 7A and 7B, a taper-shaped groove is formed on the third substrate 10. The groove on the third substrate 10 may be formed by performing etching on a single-crystal silicon substrate having a small specific resistance by using a resist film having a changing gradient. By forming the groove, the gap (space) 12 is formed between the second substrate 3 and the third substrate 10.

Then an insulation film 11 is formed by thermal oxidation. Further, etching is performed on insulation film 11 formed on a contacting surface of the third substrate 10, so that anodic bonding is established between the third substrate 10 and the borosilicate glass substrate as the second substrate 3. Further, an electrode is formed in the third substrate 10 and is connected to external voltage application means (not shown).

In the configuration of the flow rate control apparatus 1 as described above, when a voltage is applied between the electrode 8 of the second substrate 3 and the third substrate 10, an electrostatic force is generated between the electrode 8 of the second substrate 3 and the third substrate 10, thereby deforming the second substrate 3 within the gap (space) 12 in a manner such that the second substrate 3 approaches the surface of the taper-shaped groove (as illustrated in FIG. 7B). Due to the deformation of the second substrate 3, the control flow path 7 is formed (generated) so that the medical solution can flow between the first flow path 5a and the second flow path 5b (see FIG. 7B).

Herein, in the example indicated in FIGS. 6 through 7B, in order to make it easier to bend (displace) the second substrate 3, it is preferable that the thickness of the borosilicate glass (i.e., the second substrate 3) be in a range, for example, from 50 μm to 100 μm. Further, for example, when the voltage (drive voltage) is 100 V, the displacement is 5 μm and the width and the length of the formed (generated) control flow path 7 are 3 mm and 10 mm, respectively. Further, when the height difference between the transfusion container and the flow rate control apparatus 1 is 70 cm, the flow rate in the “opened valve state” indicated in FIG. 7B is 400 ml/hr and the flow rate in the “closed valve state” indicated in FIG. 7A is 30 ml/hr (when the driving cycle is 2000 Hz).

Further, in this embodiment, a case is described where the groove to be formed on the third substrate 10 is taper-shaped and is not a non-parallel shape. However, the shape of the groove is not limited to this shape. For example, the groove may have any shape such as a parallel shape (i.e., a cuboid shape). In a case where the groove has a parallel shape, it is preferable that a drive voltage to be applied be higher than that applied when the groove has a non-parallel shape.

As described above, in the flow rate control apparatus 1 according to this embodiment of the present invention, by forming (generating) the control flow path 7, it may become possible to change the fluid resistance of the transfusion (medical solution). Namely, by using the silicon substrate as the third substrate 10 and the borosilicate glass substrate as the second substrate 3, an electrostatic actuator may be configured (formed).

Due to the electrostatic force generated between the second substrate 3 and the third substrate 10, the second substrate 3 may be displaced, thereby enabling opening and closing the valve to control the flow rate. Further, the surfaces to be in contact with the transfusion fluid in the flow paths are made of single-crystal silicon, a silicon oxide film, or a silicon nitride film, which are chemical compounds of the single-crystal silicon; or the borosilicate glass only. Because of this feature, no organic matter may be dissolved into the medical solution and high safety may also be achieved.

### Pump Apparatus

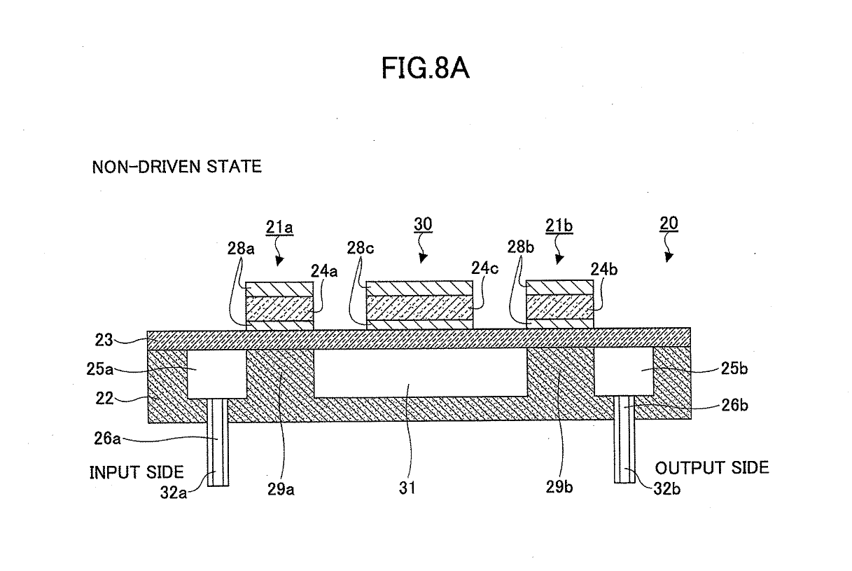

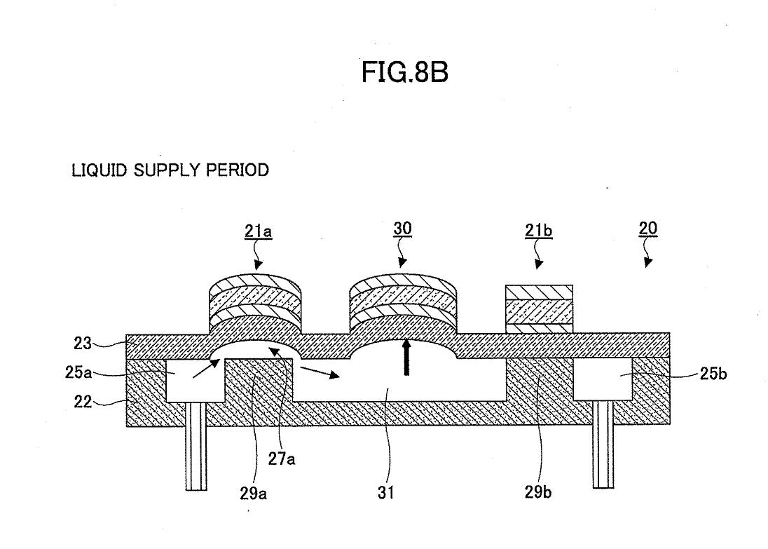

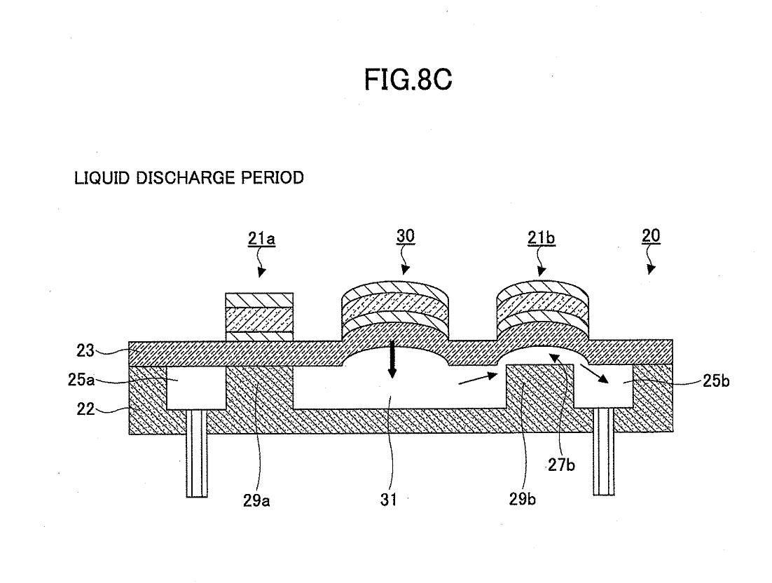

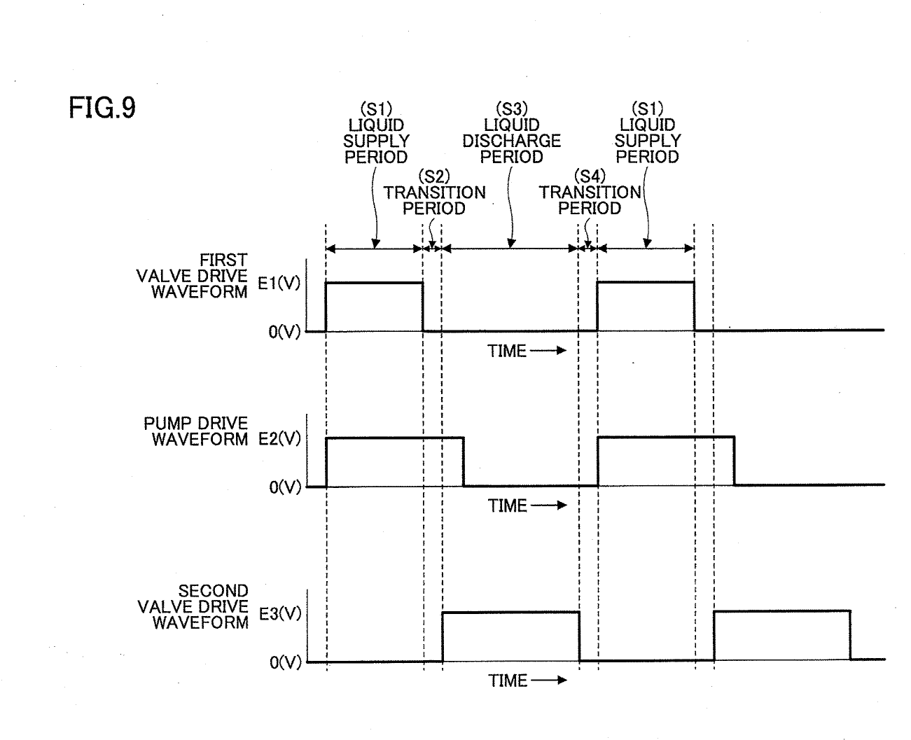

Next, a pump apparatus according to an embodiment of the present invention is described with reference to FIGS. 8A through 9. A pump apparatus in this embodiment includes a flow rate control apparatus 1 according to an embodiment of the present invention and a micro diaphragm having a piezoelectric device as a drive source. Namely, a pump apparatus 20 according to this embodiment is connected (disposed) at the middle of a medical solution injection path through which a medical solution flows from a container accommodating the medical solution and is injected into a living body, so as to adjust the flow rate of the medical solution.

As schematically illustrated in FIGS. 8A through 8C, the pump apparatus 20 includes a first substrate 22 and a second substrate 23. At least a part of the second substrate 23 is bonded to the first substrate 22. The first substrate 22 includes a first separation section 29a and a second separation section 29b which are formed in a manner such that the separation sections 29a and 29b have a thickness (height) equivalent to a thickness of the first substrate 22 where the first substrate 2 is bonded to the second substrate 23.

Further, a first flow path 25a, a liquid chamber 31, and a second flow path 25b are formed (defined) in the first substrate 22 in a manner such that the first flow path 25a and the liquid chamber 31 are separated from each other by the first separation section 29a and the liquid chamber 31 and the second flow path 25b are separated from each other by the second separation section 29b. Further, piezoelectric materials 24a, 24b, and 24c are adhered to the second substrate 23 at their respective positions on a surface of the second substrate 23, the surface of the second substrate 23 being opposite to the surface facing the first substrate 22, the respective positions corresponding to the positions of the first separation section 29a, the second separation section 29b, and the liquid chamber 31, respectively.

Further, the first substrate 22 and the second substrate 23 are not bonded to each other near the first separation section 29a and the second separation section 29b. In the description of this embodiment, the repeated descriptions of the same elements as those described in the above flow rate control apparatus 1 may be omitted.

FIGS. 8A through 8C schematically illustrate an exemplary configuration of the pump apparatus 20. As schematically illustrated in the figures, in the pump apparatus 20, the first flow path 25a, the liquid chamber 31, and the second flow path 25b are formed (defined) by performing etching on a silicon substrate as the first substrate 22.

Further, the borosilicate glass substrate as the second substrate 23 is partially bonded to the first substrate 22 based on anodic bonding. Further, above the positions corresponding to the positions of the first separation section 29a and the second separation section 29b, the electrodes 28a and 28b, respectively, are formed. Then, the piezoelectric materials 24a and 24b are adhered to (formed on) the electrodes 28a and 28b, respectively. Then electrodes 28a and 28b are formed on the piezoelectric materials 24a and 24b, respectively. In the same manner, an electrode 28c and the corresponding piezoelectric material 24c are formed. Further, a valve 21 and a pump unit 30 may be fabricated in the same manner as described above.

Herein, a first valve 21a and a second valve 21b have the same configuration as that of the valve of the flow rate control apparatus 1 described above. Namely, in the bonding between the first substrate 22 and the second substrate 23, the first substrate 22 and the second substrate 23 are strongly bonded to each other based on, for example, anodic bonding in the area other than areas near the first separation section 29a and the second separation section 29b which correspond to the areas under the piezoelectric materials 24a and 24b, respectively. On the other hand, unlike the areas where the first substrate 22 and the second substrate 23 are bonded to each other, the first substrate 22 and the second substrate 23 are not bonded to each other in the areas near the first separation section 29a and the second separation section 29b which correspond to the areas under the piezoelectric materials 24a and 24b, respectively.

Further, in the same manner, the pump unit 30 is connected to voltage application means. By applying a voltage to the piezoelectric material 24c in the pump unit 30 by the voltage application means, the piezoelectric material 24c is displaced, thereby bending the second substrate 23 adhered to the piezoelectric material 24c and changing the capacity of the liquid chamber 31. Further, the operations of the pump unit 30 are the same as those in a known micro diaphragm.

Further, a tube 32a connected to the transfusion container (medical solution container) is connected to a through hole 26a of the first flow path 25a, and a tube 32b connected to an output section (downstream side) is connected to a through hole 26b of the second flow path 25b.

FIG. 8A schematically illustrates a state where the pump apparatus 20 is not being driven. In this state, a voltage is applied to the piezoelectric material 24c provided above the liquid chamber 31, so that the liquid chamber 31 is displaced (deformed). As a result, the volume (capacity) of the liquid chamber 31 is accordingly increased, thereby enabling absorbing (increasing) the liquid in the liquid chamber 31. At the same time, a voltage is also applied to the piezoelectric material 24a of the first valve 21a, so that the valve (a control flow path 27a) is opened to decrease the fluid resistance. By doing this, the medical solution flows from the first flow path 25a on input side (upstream side) into the liquid chamber 31 (see FIG. 8B).

Next, the application of the voltage to the piezoelectric material 24a of the first valve 21a is stopped, so that the first valve 21a (i.e., the control flow path 27a) is closed so as to increase the fluid resistance. At the same time, the application of the voltage applied to the piezoelectric material 24c provided above the liquid chamber 31 is stopped, so as to increase the pressure in the liquid chamber 31 (to return to its original state).

Then, a voltage is applied to the piezoelectric material 24b of the second valve 21b so that the valve (a control flow path 27b) is opened to decrease the fluid resistance. By doing this, the medical solution flows from the liquid chamber 31 and is discharged to (injected into) the second flow path 25b on the output side (downstream side) (see FIG. 8C).

Next, the drive control of the pump apparatus 20 is described in more detail with reference to a timing chart of FIG. 9. Herein, it is assumed that drive voltages to drive the first valve 21a, the pump unit 30, and the second valve 21b are represented by symbols E1, E2, and E3, respectively.

First, in the state of FIG. 8A, the drive voltage E1 is applied to the first valve 21a to open the first valve 21a. At the same time, the drive voltage E2 is also applied to the pump unit 30 to displace (deform) an upper wall of the liquid chamber 31 to increase the volume (capacity) of the liquid chamber 31, so that the liquid solution is absorbed (transferred) from the first flow path 25a on the input side into the liquid chamber 31 (step S1: “liquid supply period”, FIG. 8B).

Next, the drive voltage E1 is set to 0 V to close the first valve 21a. On the other hand, the drive voltage E2 is maintained to keep the volume (capacity) of the liquid chamber 31 constant (step S2: “transition period”).

Next, the drive voltage E3 is applied to the second valve 21b to open the second valve 21b and the drive voltage E2 to drive the pump unit 30 is reduced down to 0 V. Due to the rigidity of the upper wall of the pump unit 30, the volume (capacity) of the liquid chamber 31 is returned to its original value or the volume (capacity) of the liquid chamber 31 may be somewhat less than the original value due to the inward bending of the second substrate 23 adhered to the piezoelectric material 24c.

Due to the decrease of the volume (capacity) of the liquid chamber 31, an internal pressure in the liquid chamber 31 is accordingly increased. At this timing, the first valve 21a is closed. Therefore, the liquid solution is discharged only into the second flow path 25b via the second valve 21b (step S3: “liquid discharge period”, FIG. 8C).

After that, an adjustment period having a predetermined time period is provided to prevent the first valve 21a and the second valve 21b from being opened at the same time. By having the adjustment period, it may become possible to prevent the liquid solution from flowing out due to external pressure (step S4: “transition period”, FIG. 8A).

By repeating the procedure from step S1 to step S4, the pump apparatus 20 pumps the transfusion fluid (medical solution) from the tube 32a on input (upstream) side to the tube 32b on output (downstream) side. Further, by exchanging the functions of the first valve 21a and the functions of the second valve 21b, the input side and the output side can be exchanged and transfusion fluid may be pumped in the direction opposite to the direction described above.

FIG. 9 illustrates a control method in which the efficiency in the flow rate of the liquid solution (medical solution) may be maximized. In this control method, in the “liquid supply period” (i.e., in the operation for supplying liquid solution into the liquid chamber 31), if the first valve 21a is not opened, liquid solution is not supplied from the first flow path 25a to the liquid chamber 31. Further, in the “liquid supply period”, if the second valve 21b is opened, liquid solution on output side may be supplied (returned) from the second flow path 25b to the liquid chamber 31. As a result, the loss of the liquid supply may be generated (increased) and the efficiency of the pumping function may be accordingly reduced. However, if the time period when the first valve 21a is not opened and/or the second valve 21b is opened is short enough when compared with the total “liquid supply period”, the medical solution may be sufficiently supplied to the liquid chamber 31 in the pumping operation.

In the same manner, in the “liquid discharge period” (i.e., in the operation for discharging liquid solution from the liquid chamber 31) if the second valve 21b is closed, liquid solution is not supplied from the liquid chamber 31 to the second flow path 25b. Further, in the “liquid discharge period”, if the first valve 21a is opened, liquid solution in the liquid chamber 31 may be supplied (returned) to the first flow path 25a (input side). As a result, the loss of the liquid supply may be generated (increased) and the efficiency of the pumping function may be accordingly reduced. However, if the time period when the second valve 21b is closed and/or the first valve 21a is opened is short enough when compared with the total “liquid discharge period”, the medical solution may be sufficiently discharged from the liquid chamber 31 to the second flow path 25b in the pumping operation.

Accordingly, even though there is a time period when the valves 21a and 21b are not set so as to efficiently supply and discharge liquid solution (medical solution), if the time period is short enough, the pump apparatus 20 may successfully perform as the pump for supplying medical solution.

The above-described pump apparatus 20 according to an embodiment of the present invention serves as not only the flow rate control apparatus but also the pump. Because of the additional feature of the pumping function, it may become possible to pump (flow) transfusion fluid (medical solution) even when there is no height difference between the transfusion container and the flow rate control apparatus or the transfusion container is disposed lower than the flow rate control apparatus.

### Medical Solution Injection System

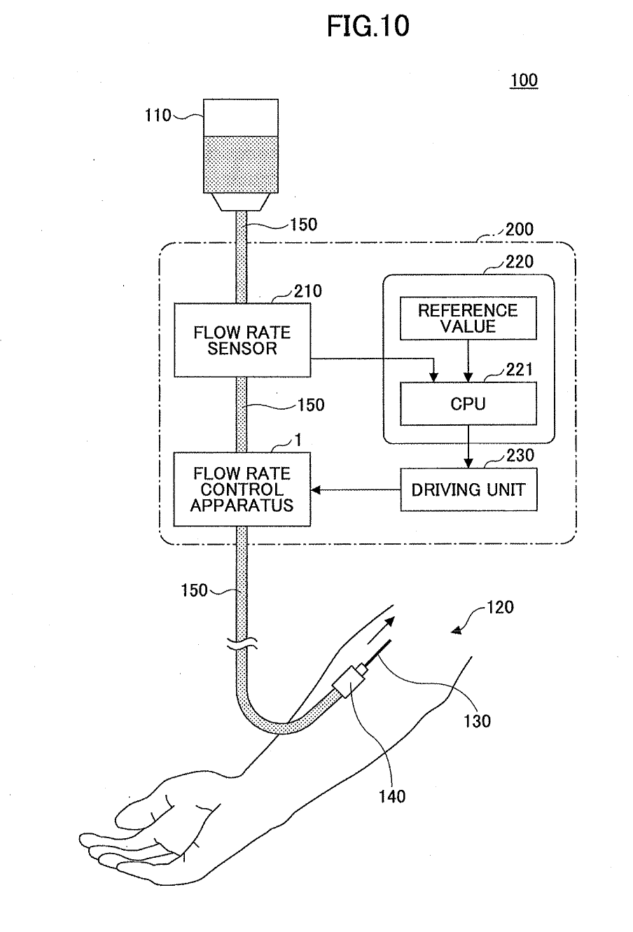

The above-described flow rate control apparatus may be included in a medical solution injection system according to an embodiment of the present invention. FIG. 10 schematically illustrates a medical solution injection system according to an embodiment of the present invention.

As illustrated in FIG. 10, a medical solution injection system 100 includes a transfusion container 110, an injection needle 130, an attachment tool 140, a medical solution injection tube 150, and a medical solution flow rate adjustment apparatus 200. The transfusion container 110 accommodates medical solution to be injected into a living body 120. The injection needle 130 is to be inserted into the living body 120 to inject the medial solution. The attachment tool 140 is provided in between the medical solution injection tube 150 and the injection needle 130 to connect them. The medical solution injection tube 150 is provided between the transfusion container 110 and the attachment tool 140 to flow (transfer) the medical solution.

The medical solution flow rate adjustment apparatus 200 is provided in the middle of the medical solution injection tube 150, and includes the flow rate control apparatus 1, a flow rate sensor 210, a control unit 220, and a driving unit 230 which serves as the voltage application means.

When medical solution is to be injected into a part (e.g., a vein) of the living body 120, the transfusion container 110 is connected to an end (input end) of the medical solution flow rate adjustment apparatus 200 via the medical solution injection tube 150. As the medical solution injection tube 150, a flexible tube having high flexibility and high self-extensibility is typically used. However, any tube having any material and any shape may alternatively used as long as medical solution can flow through the tube.

The medical solution injection tube 150 connected to the attachment tool 140 is connected to the other end (output end) of the medical solution flow rate adjustment apparatus 200 (as illustrated in FIG. 10).

In the medical solution flow rate adjustment apparatus 200, the control unit (i.e., microcomputer) 220 performs feedback control on a flow rate value detected (measured) by the flow rate sensor 210. As the flow rate sensor 210, for example, a thermal mass flow rate sensor may be used. Further, the output from the flow rate sensor 210 may be supplied in a form of an analog voltage signal or a digital output signal based on I2C, RS-232C or the like.

The control unit (i.e., microcomputer) 220 includes a CPU (Central Processing Unit) 221, an A/D converter, and a PWM (pulse width modulation) output. By having this configuration, for example, the CPU 221 compares a measured flow rate value as a signal from the flow rate sensor 210 with a predetermined reference flow rate value by performing the calculation based on the PID (proportional-integral-derivative) algorithm using three elements: deviation, integral of the deviation, and differential of the deviation to obtain a control value (such as drive force data, PWM output). The obtained control data is output (supplied) to the driving unit 230.

Based on the received control data calculated in the control unit 220, the driving unit 230 drives a power transistor to form (generate) pulses corresponding to the received control data (output voltage) to control (adjust) the flow rate to be set in the flow rate control apparatus 1. By having the configuration described above, it may become possible to control the flow rate in the flow rate control apparatus 1 in a range, for example, from 50 ml/hr to 300 ml/hr by transmitting a signal from the flow rate sensor 210 to the control unit 220 and performing the feedback control based on the PID control (algorithm).

Further, by adequately setting, for example, a value of the height difference and a width value of the flow path, it may become possible to set a flow rate value less than 50 ml/hr and/or greater than 300 ml/hr.

Further, in a medical solution injection system, by having the pump apparatus 20 according to an embodiment of the present invention instead of having the flow rate control apparatus 1, it may become possible to pump (flow) transfusion fluid (medical solution) even when there is no height difference between the transfusion container and the injection needle or the transfusion container is disposed lower than the injection needle. Further, in this case, besides the pump apparatus 20, the same components may be used and the same control method described above may be used.

According to an embodiment of the present invention, a flow rate control apparatus is provided in a middle of a medical solution injection path from a container accommodating medical solution into a living body and capable of adjusting a flow rate of the medical solution, the flow rate control apparatus including a first substrate; a second substrate, at least a part of the second substrate being bonded to the first substrate; and a piezoelectric material, in which the first substrate includes a separation section having a thickness equivalent to a thickness of the first substrate where the first substrate is bonded to the second substrate, first and second flow paths are formed in the first substrate, the first flow path and the second flow path being separated from each other by the separation section, the piezoelectric material is adhered to a position on a surface of the second substrate, the position corresponding to a position of the separation section, the surface being opposite to a surface facing the first substrate, and the first substrate is not bonded to the second substrate near the separation section.

Further, the first substrate may be made of single-crystal silicon, and the second substrate may be made of borosilicate glass.

Further, the first substrate and the second substrate may be bonded to each other based on anodic bonding in an area other than an area near the separation section.

The flow rate control apparatus may further include a voltage application unit that applies voltage to the piezoelectric material so that duty control is performed by applying a predetermined voltage by the voltage application unit.

According to an embodiment of the present invention, there is provided a flow rate control apparatus to be provided in a middle of a medical solution injection path from a container accommodating medical solution into a living body and capable of adjusting a flow rate of the medical solution. The flow rate control apparatus includes a first substrate; a second substrate, at least a part of the second substrate being bonded to the first substrate; a third substrate, at least a part of the third substrate being bonded to the second substrate; and a piezoelectric material. In the flow rate control apparatus, the first substrate includes a separation section having a thickness equivalent to a thickness of the first substrate where the first substrate is bonded to the second substrate; first and second flow paths are formed in the first substrate, the first flow path and the second flow path being separated from each other by the separation section; the piezoelectric material is adhered to a position on a surface of the second substrate, the position corresponding to a position of the separation section, the surface being opposite to a surface facing the first substrate, the piezoelectric material facing a piezoelectric material in the third substrate via a gap; and the first substrate is not bonded to the second substrate near the separation section.

According to an embodiment of the present invention, there is provided a pump apparatus to be provided in a middle of a medical solution injection path from a container accommodating medical solution into a living body and capable of adjusting a flow rate of the medical solution. The pump apparatus includes a first substrate; a second substrate, at least a part of the second substrate being bonded to the first substrate; and piezoelectric materials. In the pump apparatus, the first substrate includes first and second separation sections, each having a thickness equivalent to a thickness of the first substrate where the first substrate is bonded to the second substrate; first and second flow paths and a liquid chamber are formed in the first substrate, the first flow path and the liquid chamber being separated from each other by the first separation section, the liquid chamber and the second flow path being separated from each other by the second separation section; the piezoelectric materials are adhered to respective positions on a surface of the second substrate, the respective positions corresponding to positions of the first separation section, the second separation section, and the liquid chamber, the surface being opposite to a surface facing the first substrate; and the first substrate is not bonded to the second substrate near the first separation section and the second separation section.

According to an embodiment of the present invention, it may become possible to accurately control the flow rate with a simple configuration.

Although the invention has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

What is claimed is:

1. A flow rate control apparatus to be provided in a middle of a medical solution injection path from a container accommodating medical solution into a living body and capable of adjusting a flow rate of the medical solution, the flow rate control apparatus comprising:
a first substrate;
a second substrate, at least a part of the second substrate being bonded to the first substrate; and
a piezoelectric material;
wherein
the first substrate includes a separation section having a thickness equivalent to a thickness of the first substrate where the first substrate is bonded to the second substrate,
first and second flow paths are formed in the first substrate, the first flow path and the second flow path being separated from each other by the separation section,
the piezoelectric material is adhered to a position on a surface of the second substrate, the position corresponding to a position of the separation section, the surface being opposite to a surface facing the first substrate, and
the first substrate is not bonded to the second substrate near the separation section.

2. The flow rate control apparatus according to claim 1, wherein
the first substrate is made of single-crystal silicon, and
the second substrate is made of borosilicate glass.

3. The flow rate control apparatus according to claim 1, wherein
the first substrate and the second substrate are bonded to each other based on anodic bonding in an area other than an area near the separation section.

4. The flow rate control apparatus according to claim 1, further comprising:
a voltage application unit that applies voltage to the piezoelectric material, wherein
duty control is performed by applying a predetermined voltage by the voltage application unit.

5. A flow rate control apparatus to be provided in a middle of a medical solution injection path from a container accommodating medical solution into a living body and capable of adjusting a flow rate of the medical solution, the flow rate control apparatus comprising:
a first substrate;
a second substrate, at least a part of the second substrate being bonded to the first substrate;
a third substrate, at least a part of the third substrate being bonded to the second substrate; and
a piezoelectric material;
wherein
the first substrate includes a separation section having a thickness equivalent to a thickness of the first substrate where the first substrate is bonded to the second substrate,
first and second flow paths are formed in the first substrate, the first flow path and the second flow path being separated from each other by the separation section,
the piezoelectric material is adhered to a position on a surface of the second substrate, the position corresponding to a position of the separation section, the surface being opposite to a surface facing the first substrate, the piezoelectric material facing a piezoelectric material in the third substrate via a gap, and
the first substrate is not bonded to the second substrate near the separation section.

6. A pump apparatus to be provided in a middle of a medical solution injection path from a container accommodating medical solution into a living body and capable of adjusting a flow rate of the medical solution, the pump apparatus comprising:
a first substrate;
a second substrate, at least a part of the second substrate being bonded to the first substrate; and
piezoelectric materials;
wherein
the first substrate includes first and second separation sections, each of the sections having a thickness equivalent to a thickness of the first substrate where the first substrate is bonded to the second substrate,
first and second flow paths and a liquid chamber are formed in the first substrate, the first flow path and the liquid chamber being separated from each other by the first separation section, the liquid chamber and the second flow path being separated from each other by the second separation section,
the piezoelectric materials are adhered to respective positions on a surface of the second substrate, the respective positions corresponding to positions of the first separation section, the second separation section, and the liquid chamber, the surface being opposite to a surface facing the first substrate, and
the first substrate is not bonded to the second substrate near the first separation section and the second separation section.
